(19) United States
(12) Patent Application Publication
(10) Pub. No.: US 2008/0262413 A1
(43) Pub. Date: Oct. 23, 2008

(54) Method For Supplying Oxygenated Water To Promote Internal Healing

(76) Inventor: Daniel A. Ladizinsky, Lake Oswego, OR (US)

Correspondence Address: BROOKS KUSHMAN P.C., 1000 TOWN CENTER, TWENTY-SECOND FLOOR, SOUTHFIELD, MI, 48075, US

(21) Appl. No.: 12/103,103

(22) Filed: Apr. 15, 2008

Related U.S. Application Data
(60) Provisional application No. 60/912,696, filed on Apr. 19, 2007.

(51) Int. Cl. A61M 31/00; A61N 1/30; A61M 37/00; A61M 35/00

(52) U.S. Cl. 604/20; 604/518; 604/131; 604/290

(57) ABSTRACT

Oxygenated aqueous fluids are provided by passing aqueous hydrogen peroxide through a device containing a hydrogen peroxide decomposition catalyst. The oxygenated fluid is then used to elevate oxygen tension in a patient in need thereof.

### CROSS-REFERENCE TO RELATED APPLICATIONS

This application claims the benefit of U.S. provisional application Ser. No. 60/912,696 filed Apr. 19, 2007.

### BACKGROUND OF THE INVENTION

1. Field of the Invention

The subject invention is directed to medical oxygen therapy employing oxygenated aqueous compositions and to a method of providing such compositions.

2. Background Art

Hyperoxia, continuous or intermittent, has numerous uses in the medical field. For example, treatment of burns and ulcerated epithelial tissue has been facilitated by the use of hyperbaric pressure chambers. Oxygen dissolved in fluorinated hydrocarbons has been investigated as a means of supplying a hyperoxic state, as also has the use of ozone, particularly in solution. However, both these latter methods have extensive drawbacks, including toxicity of the fluorinated hydrocarbons as well as the exceptionally strong oxidizing ability of ozone, together with a propensity to form other highly active species such as superoxide ions. Ozone is also known to cleave ethylenic double bonds which are common in biological systems. Hyperbaric oxygen therapy is most applicable to the deeper tissues of the body, via increased blood oxygen content, and less so to the superficial tissues via external diffusion. However, hyperbaric chambers are cumbersome and expensive.

U.S. Pat. No. 5,736,582 discloses use of hydrogen peroxide as a source to generate nascent oxygen when in contact with human skin tissue by dissolving hydrogen peroxide in a non-volatile, water miscible material which stabilizes the peroxide. Oxygen is released at the skin surface by contact with hydrogen peroxide. However, the method of U.S. Pat. No. 5,736,582 allows hydrogen peroxide to directly contact the skin tissue, which is undesirable.

U.S. Pat. No. 3,996,141 discloses a method for dialysis where a semipermeable membrane contains a hydrogen peroxide catalyst, a dilute hydrogen peroxide solution is applied to one side of the membrane, and blood is contacted with the other side. Oxygen flows through the membrane into the blood. This method is only applicable to dialysis and requires an expensive and bulky dialysis machine.

U.S. Pat. No. 7,160,553 discloses the use of a crosslinked gel containing closed pores containing oxygen or another gas. When applied to tissue, the gas trapped in the pores diffuses through the gel to the tissue. The oxygen supply is tightly limited, and manufacturing is complex.

U.S. Pat. No. 5,407,685 discloses a bilayer device where each layer contains a reactant that mixes and generates oxygen once exudate or other bodily-derived material activates the reaction. The oxygen supply is limited and requires contact of the bilayer device with the tissue and exudate or bodily fluid.

It would be desirable to be able to provide oxygen therapy to other than surface areas where hyperoxia can be used to stimulate healing, to reduce inflamation, and to reduce the likelihood of infection, particularly with anaerobes.

### SUMMARY OF THE INVENTION

The invention pertains to a method for supplying oxygen to tissue, particularly to internal tissue, which avoids the drawbacks of the prior art. The method involves supplying an aqueous solution of hydrogen peroxide to an immobilized peroxide decomposition catalyst to decompose the hydrogen peroxide to form an oxygenated aqueous fluid, and supplying this fluid to a location in the body in need of oxygen therapy, particularly a state of hyperoxia.

### BRIEF DESCRIPTION OF THE DRAWINGS

FIG. 1 illustrates a patient receiving oxygenated liquid in accordance with one embodiment of the invention.

FIG. 2 illustrates one embodiment of an oxygen generating device.

FIG. 2a is an enlarged view of fibers containing H₂O₂ decomposition catalyst used in FIG. 2.

FIG. 3 illustrates a further embodiment of an oxygen generating device.

FIG. 3a is an enlarged view of the manganese dioxide H₂O₂ decomposition catalyst used in FIG. 3.

### DETAILED DESCRIPTION OF THE PREFERRED EMBODIMENT(S)

The aqueous fluid may comprise water, physiological saline, plasma, blood, etc., essentially any aqueous fluid which is tolerated by the body. Water and physiological saline are most preferred aqueous fluids. The fluids may contain numerous other substances such as pH buffers, acids or bases to adjust pH, nutrients, salts, medicaments, dyes, and the like depending on the particular application.

The hydrogen peroxide may be supplied from any source, but is preferably a dilute aqueous hydrogen peroxide source containing about 0.3% hydrogen peroxide. More generally, the hydrogen peroxide concentration is preferably less than 1% hydrogen peroxide, and most preferably from about 0.05 to 0.8 weight percent hydrogen peroxide. It is possible to employ higher strength solutions, particularly if the higher strength solution is metered into a larger aqueous stream, thus diluting it, preferably to within the ranges described above. The concentration of hydrogen peroxide when added to the circulatory system is preferably such that the oxygen formed therefrom is prevented from causing the formation of intravascular gas bubbles. For other uses, for example, irrigation of deep wounds or injection into the abdominal viscera or intestines, limited gas formation may be tolerable. This is especially true where the gas has a route of escape external to the body.

The hydrogen peroxide is supplied by a pumping system. The “pump” may be a simple gravity flow device (e.g. an I.V. bag), or may be a mechanical pump or combination thereof.

The pump delivers the hydrogen peroxide solution to a flow-through device which contains a hydrogen peroxide decomposition catalyst other than a natural exudate or bodily fluid. The catalyst should be solid and preferably immobilized. Any catalyst which decomposes hydrogen peroxide and produces either physiologically tolerable byproducts or which preferably is insoluble may be used. A suitable catalyst is manganese dioxide, which may be used in powder or granular form, in the form of fibers, or incorporated as particles or the like into polymers, e.g. polymer fibers, which are pervious to water and hydrogen peroxide. Other catalysts include metals such as silver, platinum, and gold, which may be used in the form of a porous membrane, gauze, fabric, or porous sintered material. The metal may also be plated onto a surface, for example one of polymer, glass, or metal such as stainless steel. Organic compounds are also known which decompose hydrogen peroxide, but must have exceptionally low solubility in water, i.e. be essentially insoluble. Metal catalysts and inorganic catalysts are preferred.

Most preferably, the catalyst is “immobilized”, i.e. is retained in the flow-through device. If incorporated into fibers or the like, no retaining structures may be necessary. However, if in particulate form, it may be advisable to provide a retaining device downstream from the catalyst. Such a device may consist of a paper or polymeric filter, or a microporous membrane, for example.

Upon passage through the flow-through device, the resultant aqueous stream should have the majority, preferably all the hydrogen peroxide decomposed into water and oxygen. The concentration of remaining hydrogen peroxide is preferably less than 0.2 weight percent, more preferably, in order of increasing preference, less than 0.1, 0.075, 0.05, 0.02, and 0.01 weight percent. Most preferably the concentration of hydrogen peroxide will be 0 or substantially 0 weight percent.

The flow-through device may be fitted with suitable connectors for hydrogen peroxide source and for introduction into the body. Luer lock fittings are particularly appropriate. The flow-through device may also be supplied as an integral part of a tubulature, lumen, or catheter.

The oxygen solution thus provided may be supplied to the body through any applicable medical device, for example through a lumen or catheter, tubing, optionally terminated by a sponge-like device, intravenously, or in any manner which directs the oxygenated fluid to the target area.

Dissolved oxygen in solution in saline or in water can be prepared by passing dilute hydrogen peroxide through a filter containing a catalyst that will cause the reaction of hydrogen peroxide to oxygen and water. The components may then be delivered immediately to the target tissue via a catheter system leaving the catalytic filter.

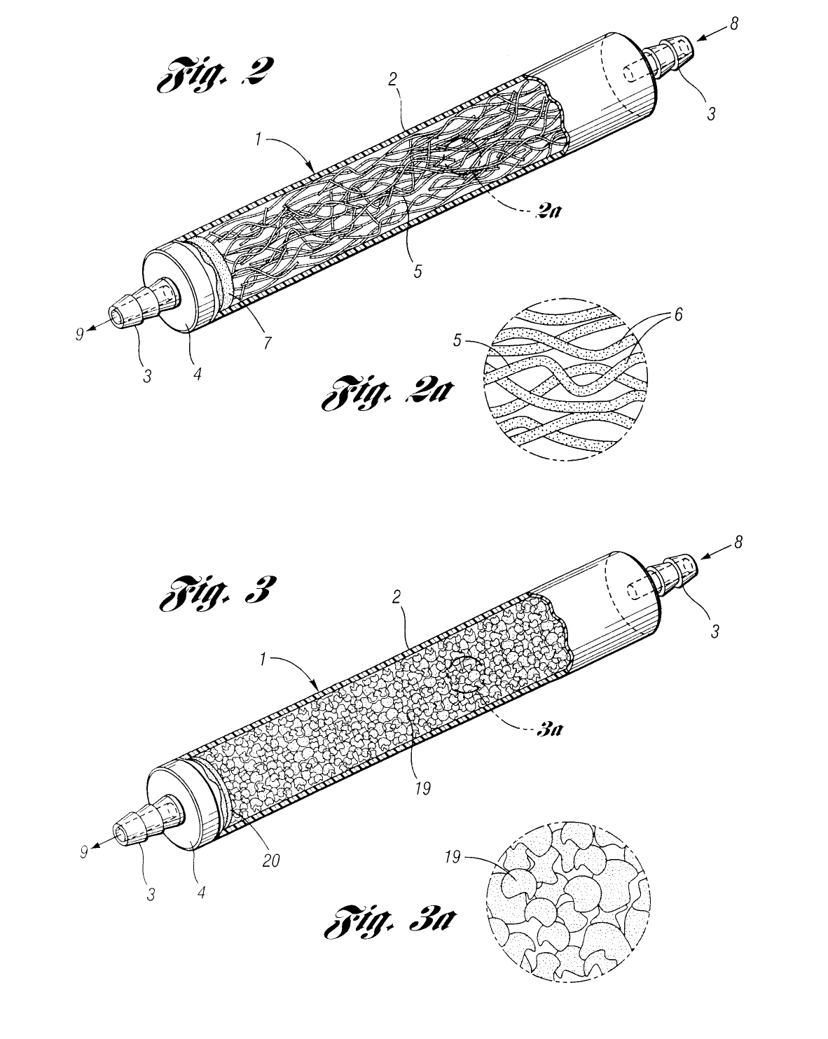

FIGS. 2 and 2a illustrate one embodiment of an oxygen generating device useful in the subject invention. The device 1 has a cylindrical wall (other cross-sections are equally possible) 2, and contains water permeable fibers 5 which contain embedded H₂O₂ decomposition particles 6. These particles may be any solid, essentially non-leachable catalyst, for example powdered silver, manganese dioxide powder, etc. The fiber is one which is permeable to water, such as a polyacrylamide, polyacrylic acid, polyvinyl alcohol, or similar homo- or copolymer. Rather than fibers, the catalyst may be incorporated into beads, rings, etc., and the polymer may be a gel-like substance as well.

The ends 4 of the cylinder have tubulatures 3 for attachment to plastic tubing or the like to convey H₂O₂ into the device (8) and to convey oxygenated water from the device (9). Near the downstream end is filter 7, which may be a membrane filter, a paper filter, a pleated filter, or the like, or as shown here, a porous sintered silver filter 7. A benefit of using the latter is that silver itself is a peroxide decomposition catalyst, so use of such a filter would help assure that all H₂O₂ has been decomposed into water and oxygen. It is also possible to dispense with the fibers 5 and expand the length and/or surface area of the sintered silver element 7 to serve as the entire H₂O₂ decomposition element.

FIGS. 3 and 3a illustrate a device similar in most aspects to FIG. 2, but containing relatively inexpensive manganese dioxide granules 19 as the H₂O₂ decomposition catalyst. The sintered silver filter 7 of FIG. 2 has been replaced with a porous membrane filter 20, which is shown in somewhat enhanced thickness for purposes of illustration.

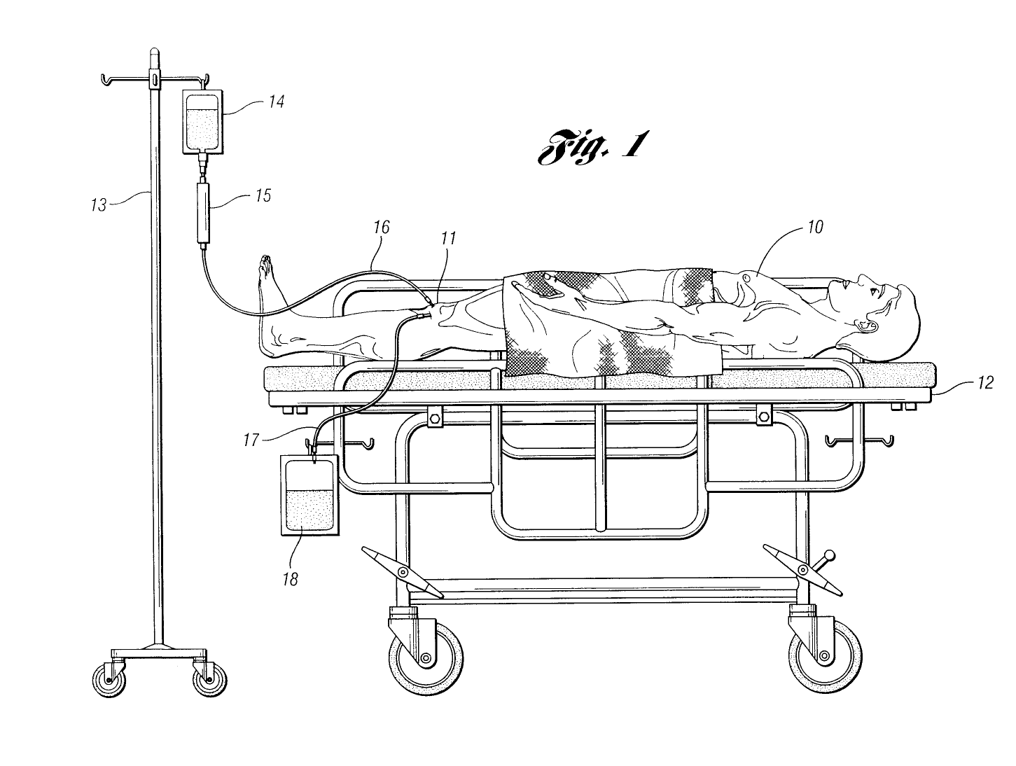

FIG. 1 illustrates a medical treatment in accordance with Example 2. The patient 10 rests on gurney 12. An IV bag 14 contains hydrogen peroxide solution in physiological saline, supported by stand 13. From the IV bag, hydrogen peroxide solution is “pumped” by gravity flow through oxygen generating device 15, which may, for example, be a device as illustrated in FIGS. 2 and 3. Oxygenated physiological saline flows through plastic tubing having a catheter at its end, into the knee joint 11. Depending upon the flow rate, it may be necessary to remove excess fluid via a second catheter-terminated tube 17 into a fluid collecting bag 18.

The applications are very broad. Others have conceived of delivering oxygen to the tissues and organs of the body by alternate oxygen carriers such as fluorocarbons or ozone. Both have problematic features and side effects that render them less than ideal for human use. It is thought that the current method will circumvent these pitfalls and deliver dissolved oxygen with minimal side effects in a simple and inexpensive manner.

Possible applications include local and systemic indications. Systemically, alternative oxygenation via nonpulmonary sources may be provided by exposing dissolved oxygen to body surfaces such as the peritoneal cavity, which will allow transport of oxygen into the tissues and bloodstream via its' large surface area (ref Chest 130(2); 402, 2006). This fluid could also be directly administered into the bloodstream in situations of cardiopulmonary compromise. Locally, there are body cavities that may be impaired or may be slow healing after injury due to low ambient oxygen, such as the articular spaces. As such, intraarticular administration could augment repair, for example after ACL repair of the knee. Short bursts of hyperoxia can be used to inhibit inflammation, which may be therapeutically useful in arthritis (ref Rheumatol Intl 26(2):142, 2005).

The healing of the abdominal viscera can be improved by topical oxygen application, which could result in fewer leaks after bowel repair and less adhesion formation (refs Bull Exp Biol Med 136(6); 582, 2003, and 137(1); 103, 2004). Also bowel ischemia can be attenuated after reperfusion (ref Brit J Surg 90(8):1015, 2003 and Shock 26(6):620, 2006) if exposed to ambient oxygen.

Any tissue that is compromised by ischemia or hypoxia can benefit from this method. Also any inflamed tissue can be cooled down by intermittent hyperoxia exposure. Also any tumor or lesion undergoing photo or radiotherapy requiring the production of singlet oxygen for it's therapeutic effect may be more easily treated if hyperoxygenated at the time of treatment.

Among the applications of the oxygen-enriched fluid, or “liquid oxygen”, are numerous categories of anatomic therapeutic targeting, as follows:

1. Topical Surface
(surface skin mucous membranes)

2. Topical Intraluminal
(mucosal surfaces—gastrointestinal, endobronchial, genitourinary)

3. Topical Intracavitary
(peritoneal, thoracic, ocular, tendon sheath, sinus, otic, cerebral intraventricular cavities)

4. Topical Intraarticular
(large and small joints by injection or infusion)

5. Intralesional Soft Tissue
(includes direct injection and iontophoresis into skin, subcutaneous, fatty, muscular, glandular and periarticular tissues—benign or malignant) This category would include use of oxygen as a sensitizer for photodynamic or radiation therapy.

6. Topical intraosseous
(into cancellous bone or fracture callus)

7. Intravascular ex vivo
(perfusate for organs awaiting transplantation or revascularization)

8. Intravascular in vivo regional
(as a method for treating ischemic tissues acutely during salvage—i.e. infusion in a leg being revascularized, in the carotid artery for brain perfusion during crossclamping, etc.)

9. Intravascular in vivo systemic
(for actual systemic oxygenation therapy in shock lung or other cardiopulmonary conditions causing inadequate systemic oxygenation)
   Thus, the method is applicable for metabolic support for healing processes/physiologic homeostasis, is anti-infective, in particular for anaerobic flora, and is a photosensitizer for photo dynamic or radiation therapy.

### EXAMPLES

### Example 1

A patient suffers from arthritis and requires steroids for its' control. He is at high risk from infection during planned colon surgery. The steroids limit his immune response and he is vulnerable to the anaerobic colonic bacteria. During the surgery, a catheter is placed that will drip a saline solution enriched in oxygen onto the area of the colon repair for several days after the surgery. This will be toxic to the anaerobic bacteria and metabolically support the early healing processes necessary for a successful outcome.

### Example 2

A patient is about to undergo regrafting of a torn anterior cruciate ligament. Previous injury surgery and the intrinsically low oxygen supply in the joint fluid limit the speed and quality of the repair process. During the surgery, a catheter is inserted in the joint that will drip oxygen enriched fluid into the joint for several days. This will help support the metabolic process allowing the graft to survive and to heal more rapidly

### Example 3

A patient has developed a bowel obstruction from adhesions formed from a prior operation. During this operation to release the obstruction, a catheter drips in an oxygen enriched fluid that will limit the formation of additional adhesions on the bowel wall that will be traumatized by even gentle surgical manipulation, reducing the risk of subsequent adhesions and obstruction.

### Example 4

A that the poorly oxygenated tissues in the center of the tumor are less affected by the radiation, as it is oxygen radicals that actually mediate the tumor cell death brought on by the radiation energy. During the radiation treatment, an injection of oxygen enriched fluid increases the oxygen tension in the poorly vascularized tumor center, thus creating a higher tumor cell kill by the radiation and a more effective cure, perhaps with even a reduced radiation dose.

While the principle use of the method of the invention is in medical treatment, the subject invention oxygen generating apparatus has industrial utility as well. For example in processes where oxygen is desired as an oxidant or reactant in an aqueous system, for example in the cleaning and/or etching of semiconductor wafers, as a sterilant in clean rooms, kitchens, and food manufacturing and processing plants and the like, the subject invention apparatus can be a substitute for a more complex oxygen delivery system employing compressed oxygen gas. This is particularly so when a source of compressed oxygen is not readily available. In such cases, the hydrogen peroxide is preferably supplied at a higher concentration, for example at 10 to 30 weight percent, and diluted just prior to use or in situ.

The references cited herein are incorporated herein by reference.

While the best mode for carrying out the invention has been described in detail, those familiar with the art to which this invention relates will recognize various alternative designs and embodiments for practicing the invention as defined by the following exemplary claims.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A method for introducing oxygen into a locale in a patient in need of oxygen therapy at said locale, comprising:
providing a flow-through immobilized peroxide decomposition catalyst device having at least one inlet for an aqueous hydrogen peroxide solution and an exit for oxygenated aqueous fluid;
introducing aqueous hydrogen peroxide solution into the inlet of said device;
flowing oxygenated aqueous fluid depleted of hydrogen peroxide to said locale.

2. The method of claim 1, wherein the hydrogen peroxide solution comprises hydrogen peroxide and substantially water or physiological saline and is introduced into said device by a pump.

3. The method of claim 1, wherein said locale is one selected from among the blood circulation system, a deep wound, the articular spaces, and the abdominal viscera.

4. An apparatus suitable for use in the method of claim 1, comprising:
a supply of aqueous hydrogen peroxide;
an immobilized peroxide decomposition device;
a conduit connecting said supply with said device, and
a delivery device suitable for delivery oxygenated fluid to the desired locale.

5. The apparatus of claim 4, wherein said pump is a gravity flow pump.

6. The apparatus of claim 4, wherein said pump is a persistaltic pump.

7. The apparatus of claim 4, where an inlet and an outlet of said device comprise luer lock fittings.

8. A method of increasing the effectiveness of phototherapy or radiotherapy to a locale wherein singlet oxygen species are created, comprising
increasing the oxygen content of said locale prior to or during exposure to light or radiation by the method of claim 1; and
exposing the locale to light or radiation to generate single oxygen.

9. The method of claim 1, wherein the oxygenated aqueous fluid is contacted with a surface mucous membrane or a intraluminal mucosal surface.

10. The method of claim 1, wherein the oxygenated aqueous fluid is introduced into one or more of an intracavitary locale selected from the group consisting of peritoneal, thoracic, ocular, tendon sheath, sinus, otic, and cerebral intraventricular cavities.

11. The method of claim 1, wherein the oxygenated aqueous fluid is introduced by injection or infusion into an intraarticular locale.

12. The method of claim 1, wherein the oxygenated aqueous fluid is introduced by direct injection or iontophoresis into intralesional soft tissue.

13. The method of claim 12, wherein the interlesional soft tissue is one or more of skin, subcutaneous tissues, fatty tissue, muscular tissue, glandular tissue, or periarticular tissue.

14. The method of claim 1, wherein the oxygenated aqueous fluid is contacted with cancellous bone or fracture callus.

15. The method of claim 1, where the oxygenated aqueous liquid is contacted ex vivo to perfuse an organ awaiting transplantation.

16. The method of claim 1, wherein the locale comprises ischemic tissues during salvage.

17. The method of claim 16, wherein the oxygen aqueous fluid is infused into an appendage being revascularized or into the brain by perfusion during crossclamping.

18. The method of claim 1, wherein the locale is one exhibiting inadequate systemic oxygenation.

19. The method of claim 18, wherein the local is a lung.
